(19) United States
(12) Patent Application Publication
(10) Pub. No.: US 2009/0093846 A1
(43) Pub. Date: Apr. 9, 2009

(54) Pre-Curved Flexible Member For Providing Dynamic Stability To A Spine

(75) Inventor: Hugh D. Hestad, Edina, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Zimmer Spine Inc., Minneapolis, MN (US)

(21) Appl. No.: 11/867,383

(22) Filed: Oct. 4, 2007

(51) Int. Cl. A61B 17/70; A61B 17/04; A61B 19/00

(52) U.S. Cl. 606/255; 606/246; 606/264; 606/301; 606/257; 128/898

(57) ABSTRACT

A pre-curved flexible member is provided for use with a dynamic stabilization system or implant to provide dynamic stability to a person's spine. In one embodiment, the pre-curved flexible member includes a pre-curved body having an outer surface and opposing ends with a curved intermediate portion extending therebetween. Each of the opposing ends includes retention features, for example notches and/or protrusions, for cooperation with retention features on a corresponding anchor member to securely retain the pre-curved flexible member therebetween.

### FIELD OF THE INVENTION

The present invention relates generally to spinal support devices and, more specifically, to a pre-curved flexible member with one or more retention features for use with a dynamic stabilization system to provide dynamic stability to a person's spine.

### BACKGROUND OF THE INVENTION

The treatment of acute and chronic spinal instabilities or deformities of the thoracic, lumbar, and sacral spine has traditionally involved the implantation of rigid rods to secure the vertebrae of a patient. More recently, flexible materials have been utilized in connection with anchor members, e.g., pedicle screws, to provide a dynamic stabilization of the spinal column. Such dynamic stabilization systems or implants typically include a flexible member positioned between pedicle screws installed in adjacent vertebrae of a person's spine.

Certain dynamic stabilization systems permit the top loading of a flexible member and connecting member between pedicle screws. One such top loading system is disclosed in U.S. Patent Application Publication No. 2002/0035366 to Walder et al., titled “Pedicle Screw For Intervertebral Support Elements”, which is expressly incorporated by reference herein in its entirety. Another top loading system is disclosed in U.S. patent application Ser. No. 11/618,943 to Hestad et al., titled “Spine Stiffening Device”, which is expressly incorporated by reference herein in its entirety. Still other dynamic stabilization systems are adapted to securely retain the flexible member between pedicle screws without the use of a connecting member.

While current dynamic stabilization systems include flexible members, these flexible members typically are of a linear and uniform cylindrical shape, which may not allow for variability in flexibility, for example, except by varying the length of the flexible member between pedicle screws. Furthermore, non-linear configurations, such as a pre-curved, or already curved, flexible member could be desirable for reducing kyphosis in patients, for example, particularly if that flexible member could be securely retained in a desired orientation. Indeed, pre-curved flexible members, which may provide variable flexibility, would be beneficial for providing surgeons with greater options in selecting the most appropriate flexible member for placement at a specific location along a patient's spine, e.g., to treat kyphosis, such selection being dictated by the desired bending movement of the flexible member at that location.

Accordingly, it would be desirable to provide a pre-curved flexible member with retention features, and which may further include a variable flexibility, for use with dynamic stabilization systems to provide dynamic stability to a person's spine that addresses these and other deficiencies of current flexible members.

### SUMMARY OF THE INVENTION

In the present invention, a pre-curved flexible member with retention features is provided for use with a dynamic stabilization system or implant to provide dynamic stability to a person's spine.

In one embodiment, the pre-curved flexible member includes a pre-curved body having an outer surface and opposing ends with a curved intermediate portion extending therebetween. In other words, the pre-curved flexible member defines a curved shaped in its relaxed state. The pre-curved body may further include a curved lengthwise central axis and the outer surface may define a circumference. Each of the opposing ends includes retention features for cooperation with an anchor member. In one example, the retention feature includes a pair of notches situated in opposing relation within the outer surface adjacent a terminal edge of respective opposing ends. In another example, the retention feature includes a pair of protrusions situated in opposing relation and extending in a direction away from the respective opposing end parallel with the curved lengthwise axis of the body. Specifically, those retention features cooperate with corresponding notches and/or protrusions on corresponding anchor members, e.g., pedicle screws. Also, it should be understood that more or less than two protrusions may be provided on the ends. In addition, the pre-curved body can further define a cylinder and may include an aperture extending lengthwise therethrough for receiving a connecting member to retain the pre-curved flexible member between pedicle screws in the dynamic stabilization system.

In another embodiment, the pre-curved flexible member further includes a first groove within the outer surface to provide the flexible member with a variable flexibility. In another embodiment, the flexible member further includes a taper in diameter of the pre-curved body or a taper in diameter of an aperture extending lengthwise through the pre-curved body. In yet another embodiment, the pre-curved body is substantially oval-shaped along its length when viewed from both ends to provide the flexible member with a variable flexibility. In still another embodiment, the pre-curved body further includes at least one first portion comprising a first material having a first elasticity and at least one second portion comprising a second material having a second elasticity greater than the first to provide the flexible member with a variable flexibility.

Accordingly, these and other various configurations of the pre-curved flexible member allow the pre-curved flexible member to be securely retained in a desired orientation and, thus, can be beneficial, for example, in treating kyphosis in a patient. The pre-curved flexible member may further include a variable flexibility that can allow for easier bending of the pre-curved flexible member in one direction relative to another, as compared to conventional linear flexible members which have equal bending force in all directions.

One or more of the flexible members can be utilized in a method for stabilizing a patient's spine. In one embodiment, the method includes providing a plurality of implants. Each of the implants includes a flexible member having a pre-curved body including an outer surface and opposing ends with a curved intermediate portion extending therebetween. Each of the opposing ends has a retention feature for cooperation with an anchor member to limit rotation of the pre-curved body. The retention feature at each of the respective opposing ends is selected from one or a combination of (a) at least one notch provided within the outer surface of the respective opposing end and/or (b) at least one protrusion extending in a direction away from the respective opposing end. Next, at least one implant is selected from the plurality of implants based on the condition of the patient. Then, the selected implant is implanted into the patient.

By virtue of the foregoing, there is provided a pre-curved flexible member with retention features for use with a dynamic stabilization system to provide dynamic stability to a person's spine.

The features and objectives of the present invention will become more readily apparent from the following Detailed Description taken in conjunction with the accompanying drawings.

### BRIEF DESCRIPTION OF THE DRAWING

The accompanying drawings, which are incorporated in and constitute a part of this specification, illustrate an embodiment of the invention and, together with the general description of the invention given above, and detailed description given below, serve to explain the invention.

FIG. 1A is a side elevational view of a dynamic stabilization system including top loading anchor members inserted into the spinal column and a connecting member, and one embodiment of a pre-curved flexible member with retention features secured between the anchor members;

FIG. 1B is a side elevational view of a dynamic stabilization system including top loading anchor members inserted into the spinal column and a connecting member, and another embodiment of a pre-curved flexible member with retention features secured between the anchor members;

FIG. 2A is a perspective view of the pre-curved flexible member of FIG. 1A;

FIG. 2B is a perspective view of the pre-curved flexible member of FIG. 1B;

FIGS. 2C, 3 and 4 are perspective views of other embodiments of a pre-curved flexible member;

FIG. 4A is a cross-sectional view of the pre-curved flexible member of FIG. 4 taken along the line 4A-4A;

FIG. 5 is a perspective view of another embodiment of a pre-curved flexible member;

FIG. 5A is a cross-sectional view of the pre-curved flexible member of FIG. 5 taken along the line 5A-5A;

FIG. 6 is a perspective view of another embodiment of a pre-curved flexible member;

FIG. 6A is a cross-sectional view of the pre-curved flexible member of FIG. 6 taken along the line 6A-6A;

FIG. 7 is a perspective view of another embodiment of a pre-curved flexible member;

FIG. 7A is a cross-sectional view of the pre-curved flexible member of FIG. 7 taken along the line 7A-7A;

FIGS. 8-23 are perspective views of various embodiments of a pre-curved flexible member;

FIG. 24 is a perspective view of another embodiment of a pre-curved flexible member;

FIG. 24A is a cross-sectional view of the pre-curved flexible member of FIG. 24 taken along the line 24A-24A;

FIGS. 25-27 are perspective views of other embodiments of a pre-curved flexible member;

FIG. 27A is a cross-sectional view of the pre-curved flexible member of FIG. 27 taken along the line 27A-27A;

FIG. 28 is a perspective view of another embodiment of a pre-curved flexible member;

FIG. 29 is a cross-sectional view of another embodiment of a pre-curved flexible member similar to the pre-curved flexible member of FIG. 28;

FIG. 30A is a disassembled, perspective view of an embodiment of a connecting member with the pre-curved flexible member of FIGS. 1A and 1B for use in a dynamic stabilization system;

FIG. 30B is a partially disassembled view of a dynamic stabilization system utilizing the connecting member and pre-curved flexible member shown in FIG. 30A and top loading anchor members; and

FIG. 30C is a cross-sectional view of the assembled dynamic stabilization system of FIG. 30B.

### DETAILED DESCRIPTION OF THE INVENTION

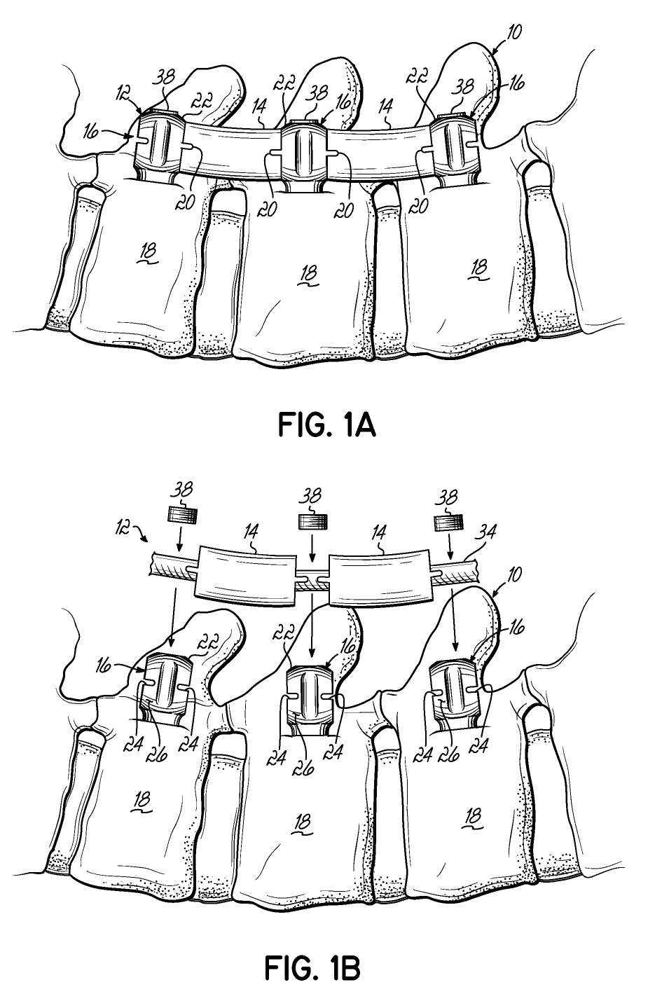

FIGS. 1A and 1B illustrate cut-away sections of a spine 10 having a dynamic stabilization system or implant 12 implanted therein. The systems 12 of FIGS. 1A and 1B, include pre-curved flexible members 14 securely retained between anchor members 16, for example, pedicle screws, installed in adjacent vertebrae 18 of the spine 10.

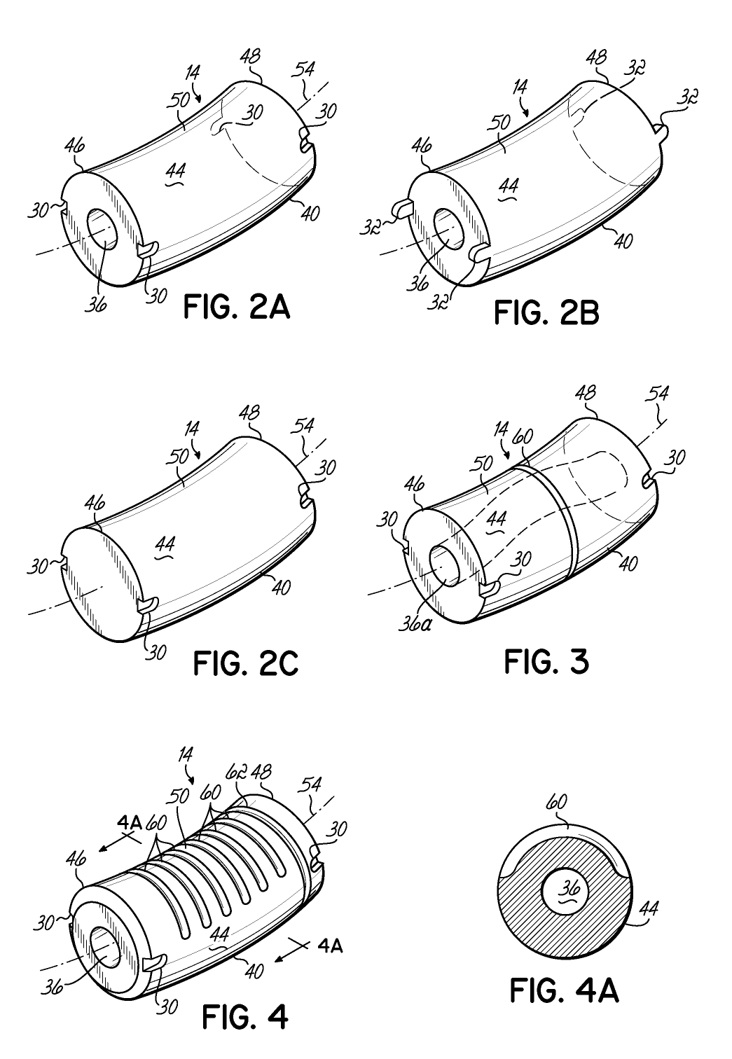

The anchor members 16 of FIGS. 1A and 1B generally illustrate top loading anchor members 16 that retain the pre-curved flexible members 14 therebetween. More specifically, each anchor member 16 in FIG. 1A includes anchor member retention features or protrusions 20, which generally extend in a direction away from each anchor member head 22, whereas each anchor member 16 in FIG. 1B includes retention features or notches 24 provided within the outer surface 26 of each anchor member head 22. These anchor member retention features 20, 24 cooperate with flexible member retention features, i.e., corresponding notches 30 and/or protrusions 32 (FIGS. 2A and 2B), on the pre-curved flexible members 14, as further discussed below. One such top loading type anchor member or screw that may be modified to include protrusions 20 or notches 24, for example, is disclosed in U.S. Patent Application Publication No. 2002/0035366 to Walder et al., titled “Pedicle Screw For Intervertebral Support Elements”, which is expressly incorporated by reference herein in its entirety.

With further reference to FIG. 1B, a connecting member 34 (not shown in FIG. 1A) is passed through an aperture 36 (FIGS. 2A and 2B) in the pre-curved flexible member 14. Such connecting member 34 then is top loaded and secured within a top portion of each anchor member 16 by threadable cap members 38. The connecting member 34 can be passed through the aperture 36 during or prior to implantation in a patient, or preformed or coupled to the pre-curved flexible member 14 to form a unitary structure during manufacture of the dynamic stabilization system 12. Once secured, that connecting member 34, along with the respective retention features 20, 24, 30, 32 helps retains the pre-curved flexible member 14 between the anchor members 16 at a desired orientation while cooperating with the pre-curved flexible member 14 for permitting mobility of the spine 10. The retention features help the pre-curved flexible member maintain that desired orientation.

In accordance with embodiments of the present invention, the pre-curved flexible members 14 of FIGS. 1A and 1B, as best shown in FIGS. 2A and 2B, respectively, include a pre-curved body 40 including an outer surface 44 and opposing ends 46 and 48 with a curved intermediate portion 50 extending therebetween. The pre-curved body 40 has a cylindrical shape and includes a curved lengthwise central axis 54, and the outer surface 44 further defines a circumference. Each of the opposing ends 46, 48 includes flexible member retention features 30 (FIG. 2A), 32 (FIG. 2B) for cooperation with anchor members 16. More specifically, with reference to FIG. 2A, the flexible member retention feature includes a pair of notches 30 situated in opposing relation within the outer surface 44 adjacent a terminal edge of respective opposing ends 46, 48 for cooperation with corresponding anchor member retention feature 20. With reference to FIG. 2B, the flexible member retention feature includes a pair of protrusions 32 situated in opposing relation and extending in a direction away from the respective opposing end 46, 48, parallel with the curved lengthwise axis 54 of the pre-curved body 40 for cooperation with corresponding anchor member retention features 24. Although two notches 30 or protrusions 32 are shown on each opposing end 46, 48 of FIGS. 2A and 2B, respectively, it should be understood that more or less than two may be provided such as for cooperation with respective anchor member retention features 20, 24. In addition, combinations of notches 30 and protrusions 32 are contemplated. Also, the retention features 20, 24, 30, 32 may vary in shape and size as is desired.

FIG. 2C, which is similar to FIG. 2A, depicts another embodiment of pre-curved flexible member 14 wherein the pre-curved flexible member 14 of FIG. 2C is devoid of aperture 36 and, thus, does not require corresponding connecting member 34. Instead, the pre-curved flexible member 14 can be directly top loaded into anchor members 16 and securely retained by anchor member retention features 20, for example. In addition, it should be understood by one of ordinary skill in the art that the pre-curved flexible members 14 of FIGS. 2A and 2B may be used in a system 12 without connecting member 34. The pre-curved flexible member 14 also may be provided in varying degrees of curvature and also lengths, e.g., twelve-inch lengths, so that a surgeon can cut, or shape, the pre-curved flexible member 14 to fit between opposing anchor members 16 along a specific section of spine 10, as well as to accommodate a desired bending movement of the pre-curved flexible member 14.

Orientation of the pre-curved flexible member 14, e.g., lateral versus anterior/posterior positioning relative to the spine 10 is determined by the desired bending movement of the selected pre-curved flexible member 14 at that specific section of spine 10. In other words, orientation of the pre-curved flexible member 14 is generally determined based upon the needs of the patient, with the pre-curved flexible member 14 of the present invention allowing for tailoring thereof on a patient-by-patient basis. In addition, although the pre-curved flexible member 14 is illustrated as being cylindrical, it should be understood by one having ordinary skill in the art that other desired shapes, for example, square, rectangular, oval, etc. may be utilized.

FIG. 3 depicts another embodiment of pre-curved flexible member 14, which includes pre-curved body 40 of a cylindrical shape having outer surface 44 and opposing ends 46, 48. The pre-curved body 40 further includes a curved intermediate portion 50 extending between opposing ends 46, 48. The pre-curved body 40 also includes curved lengthwise central axis 54 and the outer surface 44 further defines a circumference. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 further includes a groove 60, which is optional, substantially directly in-between the ends 46, 48. The groove 60 is situated perpendicular to curved lengthwise axis 54 of the pre-curved body 40 and extends around the full circumference. Although, the groove 60 is shown directly in-between the ends 46, 48, it should be understood that it could be provided closer to either of the first or second ends 46, 48 as desired. The pre-curved flexible member 14 further includes optional multi-curved aperture 36a generally defining an s-shape and extending lengthwise through the pre-curved body 40 for receiving the connecting member 34.

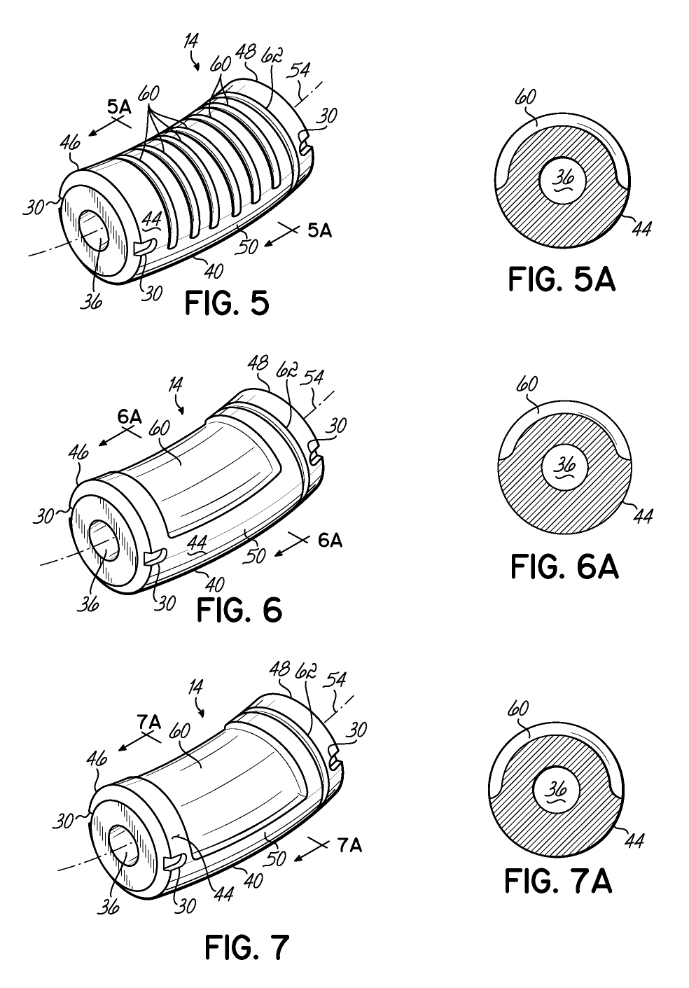

FIGS. 4 and 5 depict additional embodiments of pre-curved flexible member 14 which include pre-curved body 40 of a cylindrical shape having outer surface 44 and opposing ends 46, 48 with curved intermediate portion 50 extending therebetween. The pre-curved body 40 also includes curved lengthwise central axis 54 and the outer surface 44 further defines a circumference. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 further includes a plurality of spaced-apart grooves 60 therein situated perpendicular to the curved lengthwise axis 54 of the pre-curved body 40. As best shown in FIGS. 4A and 5A, respectively, the grooves 60 of FIG. 4 extend around no more than half the circumference of the pre-curved body 40 whereas the grooves 60 of FIG. 5 extend around approximately half the circumference of the pre-curved body 40. Optional groove 62 is further situated perpendicular to the lengthwise axis 54 of the pre-curved body 40 proximate the second end 48, and extends around the full circumference of the pre-curved body 40. The grooves 60, 62 provide the pre-curved flexible member 14 with a variable flexibility as discussed further below. The pre-curved flexible member 14 further includes aperture 36 extending lengthwise through the pre-curved body 40 for receiving the connecting member 34.

While six grooves 60 are shown in FIGS. 4 and 5, it should be understood that more or less than six grooves 60 may be provided. Also, the spacing between grooves 60 may be equal, unequal, or a mixture thereof as is desired. And, although grooves 60 are shown as being perpendicular to the curved lengthwise axis 54, one or more grooves 60 may be slightly askew or substantially perpendicular thereto. Furthermore, even though the grooves 60, 62 are shown, for example, as extending around less than half the circumference or around the entire circumference, respectively, variations of the length thereof are readily understood. In addition, optional groove 62, which is situated near the second end 48, can allow the surgeon to securely grip, or hold, the pre-curved flexible member 14, for example, with a tool (not shown), such as a clamp, so that the pre-curved flexible member 14 can be cut to a desired size. It should be understood that the optional groove 62 may be provided near the first end 48 or at both the first and second ends 46, 48.

Orientation of the pre-curved flexible member 14, e.g., inferior or superior positioning of one end 46, 48 relative to the spine 10 and/or lateral versus anterior/posterior positioning of grooves 60 similarly is determined by the desired bending movement of the selected pre-curved flexible member 14 at that specific section of spine 10. With respect to the bending movement of the pre-curved flexible member 14, the size, i.e., depth, width, and length, of the groove 60, 62 as well as the number thereof and the degree of curvature generally determine the degree and variability of flexibility for the pre-curved flexible member 14.

For example, the pre-curved flexible member 14 will both flex and extend more easily at the location of optional groove 62, which again extends around the full circumference, as compared to areas devoid of such groove 62. And, with respect to grooves 60, both individually and collectively, the pre-curved flexible member 14, when the ends 46, 48 are forced further in a direction toward grooves 60, will flex more easily as compared to areas that are devoid of such grooves 60. In contrast, when the ends 46, 48 are forced in a direction away from grooves 60, the pre-curved flexible member 14 may not experience the same ease of flexibility. Such differential in flexion as compared to extension can be generally attributed to the grooves 60 extending around no more than half the circumference. Therefore, if grooves 60 of the pre-curved flexible member 14 are located anterior relative to the spine 10, the pre-curved flexible member 14 can allow for easier bending anteriorly as compared to posteriorly or laterally. Consequently, the pre-curved flexible member 14 could be rotated 180 degrees, for example, and then the anterior and lateral bending would require more force to allow similar ease of bending in contrast to posterior bending.

The surgeon implanting the dynamic stabilization system 12 can selectively take advantage of the varying flexibility of pre-curved flexible member 14 to treat an indication or condition in the patient. The surgeon can be provided with a plurality of pre-constructed systems 12 that have pre-curved flexible members 14 with varying flexibility characteristics, or, alternatively, be provided with a variety of pre-curved flexible members 14 with varying flexibility characteristics any one of which can be incorporated into a system 12 that is constructed during the surgical procedure.

FIG. 6 depicts another embodiment of pre-curved flexible member 14, which includes pre-curved body 40 of a cylindrical shape having outer surface 44 and opposing ends with curved intermediate portion extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and the outer surface 44 further defines a circumference and includes groove 60 therein of a width greater than half the length of the pre-curved body 40. The groove 60 further is situated perpendicular to the curved lengthwise axis 54 of the pre-curved body 40 and, as best shown in FIG. 6A, extends around no more than half the circumference of the pre-curved body 40. Optional groove 62 is also situated perpendicular to the curved lengthwise axis 54 of the pre-curved body 40 proximate the second end 48, and extends around the full circumference of the pre-curved body 40. The pre-curved flexible member 14 further includes optional aperture 36 extending lengthwise through the pre-curved body 40 for receiving connecting member 34.

FIGS. 7 and 7A show a pre-curved flexible member 14 similar to FIGS. 6 and 6A, respectively, that has groove 60 of a width greater than half the length of the pre-curved body 40 and optional groove 62. However, groove 60, as best shown in FIG. 7A, extends around approximately half the circumference of the pre-curved body 40. The pre-curved flexible member 14 shown in FIGS. 6 and 7 provides a cross-sectional and lengthwise variability in flexibility that is dependent upon its configuration.

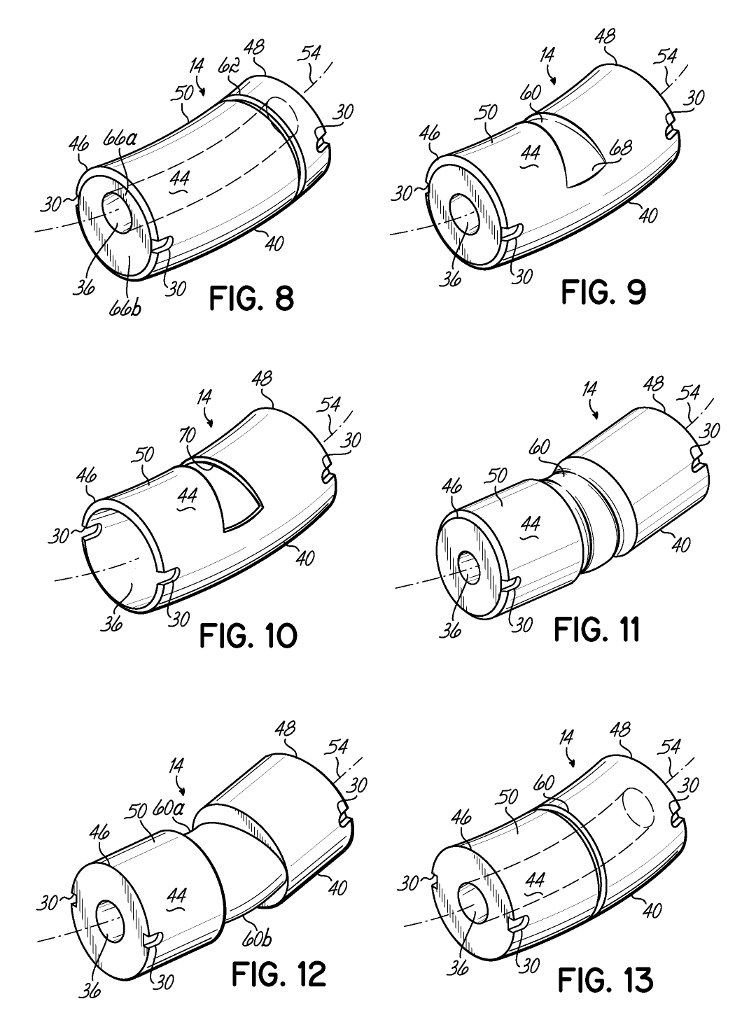

FIG. 8 depicts another embodiment of pre-curved flexible member 14, which includes pre-curved body 40 of a cylindrical shape having outer surface 44 and opposing ends 46, 48 with curved intermediate portion 50 extending therebetween. The pre-curved body 40 also includes curved lengthwise central axis 54 and the outer surface 44 further defines a circumference. Each of the opposing ends 46, 48 includes a pair of notches 30 situated in opposing relation within the outer surface 44 adjacent a terminal edge of respective opposing ends 46, 48 for cooperation with corresponding anchor member retention features 20. Optional groove 62 is situated perpendicular to the curved lengthwise axis 54 of the pre-curved body 40 proximate the second end 48, and extends around the full circumference of the pre-curved body 40. The pre-curved flexible member 14 further includes optional aperture 36 extending lengthwise through the pre-curved body 40 for receiving connecting member 34. Aperture 36 is positioned offset from the curved lengthwise axis 54. This offset positioning affords the pre-curved flexible member 14 with opposing lengthwise areas 66a and 66b, which are disposed about the aperture 36, that differ in thicknesses and, thus, provide the pre-curved flexible member 14 with variable flexibility. It should be understood that the thinnest area 66a is the most flexible with the thickest area 66b being the least flexible.

FIG. 9 depicts another embodiment of pre-curved flexible member 14, which includes pre-curved body 40 of a cylindrical shape having outer surface 44 and opposing ends with curved intermediate portion extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and the outer surface 44 further defines a circumference and has groove 60 therein with opposing flared ends 68 (only one shown). The groove 60 is situated perpendicular to the curved lengthwise axis 54 of the pre-curved body 40 and extends around no more than half the circumference. Although the groove 60 is shown substantially directly in-between the ends 46, 48, it should be understood that it could be provided closer to either of the first or second ends 46, 48 as desired. The pre-curved flexible member 14 further includes optional aperture 36 extending lengthwise through the pre-curved body 40 for receiving connecting member 34.

FIG. 10 depicts another embodiment of pre-curved flexible member 14, which is similar to FIG. 9, except that pre-curved body 40 is tubular-shaped. In other words, the diameter of optional aperture 36 of FIG. 9 is greatly enlarged. In addition, the groove 60 of FIG. 9 now cooperates with aperture 36 to define, as shown in FIG. 10, an opening 70 in pre-curved body 40.

FIG. 11 depicts yet another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54, and the outer surface 44 further defines a circumference and has groove 60 therein substantially directly in-between the ends 46, 48. The groove 60 is situated perpendicular to the lengthwise axis 54 of the pre-curved body 40, extends around the full circumference, and has a width greater than about one-third and less than about two-thirds, e.g., about one half, the full length of the pre-curved body 40 such that the pre-curved body 40 substantially defines a dumbbell shape. Although, the groove 60 is shown directly in-between the ends 46, 48, it should be understood that it could be provided closer to either of the first or second ends 46, 48 as desired. The depicted configuration allows the ends 46, 48 to move, e.g., flex, generally independently of one another. The pre-curved flexible member 14 further includes optional aperture 36 extending lengthwise through the pre-curved body 40 for receiving the connecting member 34.

FIG. 12 depicts yet another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and the outer surface 44 further defines a circumference. The pre-curved body 40 further includes a pair of grooves 60a and 60b situated in opposing relation, with each extending around less than half the circumference of the pre-curved body 40. Each groove 60a, 60b increases in depth in a direction from opposing ends 46, 48 to a center of the groove 60a, 60b to define crescent-shaped grooves. Such grooves 60a, 60b, are situated substantially directly in-between the ends 46, 48 with the curved intermediate portion 50 being substantially oval-shaped when viewed in cross-section perpendicular to the curved lengthwise axis 54 of the pre-curved body 40. This configuration, similar to FIG. 11, allows the ends 46, 48 to move generally independently of one another with the exception that the pre-curved flexible member 14 does not yield an equal bending force in all directions collectively about grooves 60a, 60b. The pre-curved flexible member 14 further includes optional aperture 36 extending lengthwise through the pre-curved body 40 for receiving the connecting member 34.

FIG. 13 depicts yet another embodiment of pre-curved flexible member 14, which is similar to FIG. 11. However, groove 60 is much smaller in width as compared to groove 60 of FIG. 11, which has a width greater than about one-third and less than about two-thirds the full length of pre-curved body 40. This smaller width limits the range of motion of the pre-curved flexible member 14 about the groove 60.

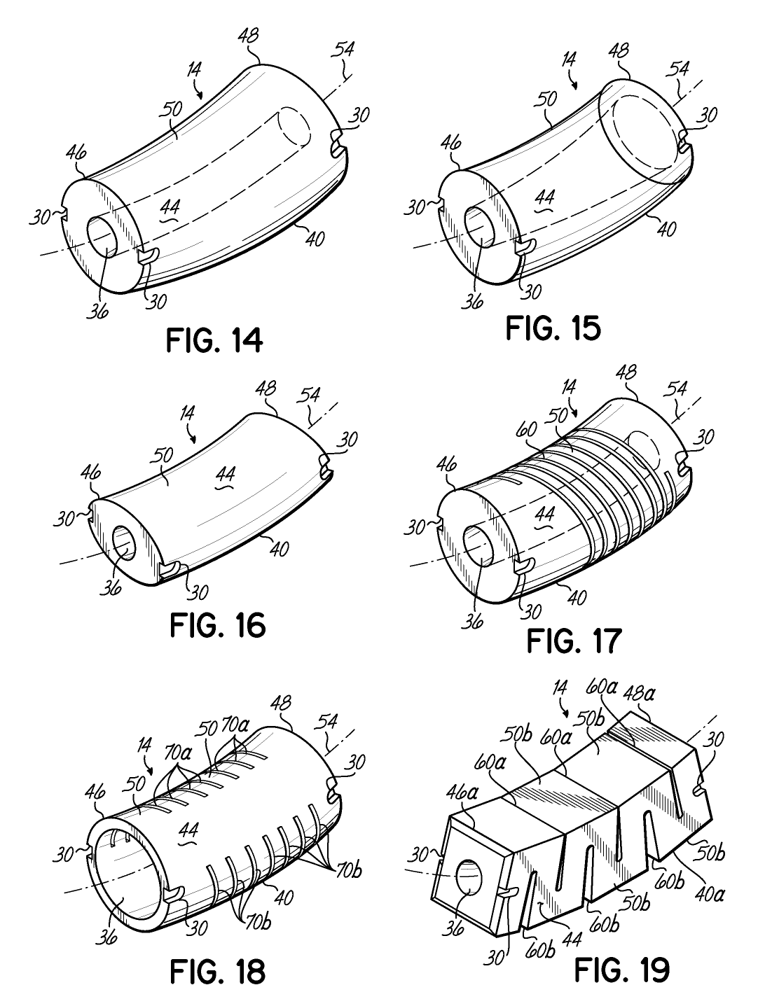

FIG. 14 depicts another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54. The pre-curved flexible member 14 further includes optional aperture 36 extending lengthwise through the pre-curved body 40 for receiving the connecting member 34. The aperture 36 maintains a constant diameter while the pre-curved flexible member 14 includes a taper in the diameter of the pre-curved body 40 from the second end 48 to the first end 46 to provide the pre-curved flexible member 14 with a variable flexibility. Specifically, with the tapered configuration, the pre-curved body 40 decreases in thickness from the second end 48 towards the first end 46 thereby defining a flexibility gradient along its length. It should be understood that the thinnest area, i.e., the first end 46, is the most flexible area with the thickest area, i.e., the second end 48, being the least flexible.

FIG. 15 depicts another embodiment of pre-curved flexible member 14, which is a variation of the embodiment depicted in FIG. 14. Rather than including a taper in diameter of the pre-curved body 40, the pre-curved flexible member 14 includes a taper in the diameter of the aperture 36 as it extends lengthwise through the pre-curved body 40 from the second end 48 to the first end 46. The pre-curved body 40 maintains a constant diameter. With this tapered configuration, the pre-curved body 40 decreases in thickness from the first end 46 towards the second end 48 similarly defining a flexibility gradient along its length to provide the pre-curved flexible member 14 with a variable flexibility.

FIG. 16 depicts another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 is substantially oval-shaped along its length when viewed from both ends 46, 48 to provide the pre-curved flexible member 14 with a variable flexibility. The pre-curved body 40 further includes curved lengthwise central axis 54 and optional aperture 36 extending lengthwise through the pre-curved body 40 for receiving the connecting member 34. The pre-curved flexible member 14 shown in FIG. 16 does not yield an equal bending force along its length in all directions but rather provides variable flexibility, which is dependent upon its oval-shaped configuration.

FIG. 17 depicts yet another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54, and the outer surface 44 further defines a circumference and has groove 60 which spirals about a portion of the pre-curved body 40 in a direction generally towards the second end 48. It is contemplated that the spacing between the turns of groove 60 may be increased or decreased as the groove 60 spirals towards the second end 48, for example, to provide the pre-curved flexible member 14 generally with a cross-sectional and lengthwise flexibility gradient. Other spiral variations may be provided. The spiral configuration of the pre-curved flexible members 14 can allow for more consistent bending forces in all directions and can provide a desirable spring-action effect. The pre-curved flexible member 14 further includes optional aperture 36 extending lengthwise through the pre-curved body 40 for receiving the connecting member 34.

FIG. 18 depicts another embodiment of pre-curved flexible member 14, which is a variation of the embodiment depicted in FIG. 17. In this embodiment, the pre-curved body 40 is tubular-shaped. In other words, the diameter of optional aperture 36 of FIG. 18 is greatly enlarged. In addition, the groove 60 of FIG. 9, which spirals about the body, now generally provides a discontinuous spiral that cooperates with aperture 36 so as to define, as shown in FIG. 10, openings 70a and 70b in pre-curved body 40.

FIG. 19 depicts another embodiment of pre-curved flexible member 14, which includes pre-curved body 40a having outer surface 44 and defining a rectangular prism that includes opposing first and second rectangular bases 46a, 48a connected by four rectangular lateral faces 50b extending therebetween. The pre-curved body 40a further includes curved lengthwise central axis 54 and first plurality of grooves 60a and a second plurality of grooves 60b. The grooves 60a, 60b are spaced offset from one another and situated in and along the full width of opposing lateral faces 50b of the pre-curved body 40a perpendicular to the curved lengthwise axis 54 such that the pre-curved body 40a is substantially serpentine-shaped to provide the pre-curved flexible member 14 with a variable flexibility. Each of the rectangular bases 46a, 48a includes a pair of notches 30 situated in opposing relation within the outer surface 44 adjacent a terminal edge of respective opposing rectangular bases 46a, 48a for cooperation with corresponding anchor member retention features 20. The pre-curved body 40a further includes optional aperture 36 extending lengthwise through the pre-curved body 40a for receiving the connecting member 34.

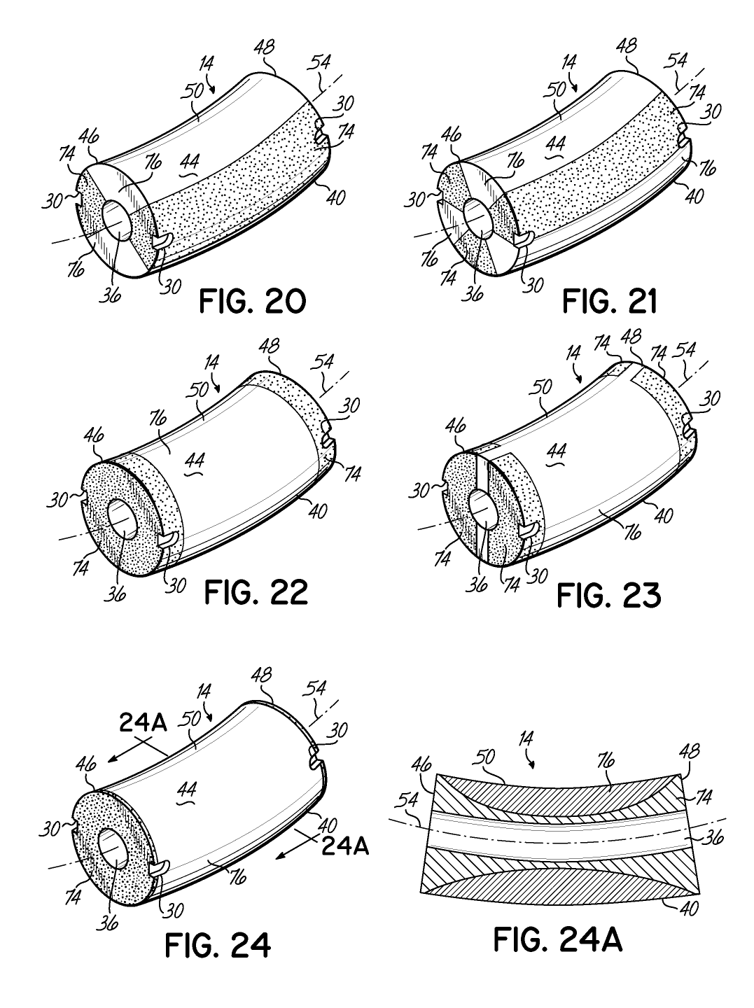

FIG. 20 depicts another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and a plurality of first portions 74, i.e., two first portions, including a first material, for example, a first polymeric material, having a first elasticity and a plurality of second portions 76, i.e., two second portions, including a second material, for example, a second polymeric material, having a second elasticity greater than the first to provide the pre-curved flexible member 14 with a variable flexibility. The first material may be a different material than the second material. In addition, the plurality of first portions 74 as well as the plurality of second portions 76 may have different elasticities and/or may be composed of different material, including, for example, polymeric, metal, or ceramic materials having a range of elasticities from flexible, to rigid, to semi-rigid that result in a pre-curved flexible member 14 that can be tailored to a particular surgical application.

Additionally, the first and second portions 74, 76 extend along the length of the pre-curved body 40 and are alternately spaced symmetrically about the axis 54 of the pre-curved body 40. Although not shown, the first and second portions 74, 76 could be spaced asymmetrically about the axis 54. The pre-curved flexible member further includes aperture 36 extending lengthwise through the pre-curved body 40 for receiving the connecting member 34. While two first and two second portions 74, 76 are illustrated in FIG. 20, in its simplest form one first portion 74 and one second portion 76 can be provided. In addition, the width of the portions 74, 76 may be varied. The construction of pre-curved flexible member 14 shown in FIG. 20 provides a cross-sectional flexibility gradient that is dependent upon the elastomeric characteristics of the first and second portions 74, 76. The surgeon implanting the dynamic stabilization system 12 can selectively take advantage of the varying elasticity of the portions 74, 76 of pre-curved flexible member 14 to treat an indication or condition in the patient. The surgeon can be provided with a plurality of pre-constructed systems 12 that have pre-curved flexible members with varying flexibility characteristics, or, alternatively, be provided with a variety of pre-curved flexible members with varying flexibility characteristics any one of which can be incorporated into a system 12 that is constructed during the surgical procedure.

FIG. 21 depicts another embodiment of pre-curved flexible member 14, which is similar to FIG. 20. The pre-curved flexible member 14 of FIG. 21 similarly includes the plurality of first portions 74 including a first material having a first elasticity and the plurality of second portions 76 including a second material having a second elasticity greater than the first to provide the pre-curved flexible member 14 with variable flexibility. However, rather than two first portions 74 and two second portions 76 as in FIG. 20, the pre-curved flexible member 14 of FIG. 21 includes three first portions 74 and three second portions 76. The pre-curved flexible member 14 further includes optional aperture 36 extending lengthwise through the pre-curved body 40 for receiving connecting member 34. The construction of pre-curved flexible member 14 shown in FIG. 21 provides a cross-sectional flexibility gradient that is dependent upon the elastomeric characteristics of the first and second portions 74, 76.

FIG. 22 depicts another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and one each of two first portions 74 defining the first and second ends 46, 48, respectively, and a second portion 76 defining the curved intermediate portion 50. The first portions 74 include a first material having a first elasticity and the second portion 76 includes a second material having a second elasticity greater than the first to provide the pre-curved flexible member 14 with variable flexibility. The pre-curved flexible member 14 further includes optional aperture 36 extending lengthwise through the pre-curved body 40 for receiving connecting member 34. The material and/or elasticity thereof for the first portions 74 may be the same or different. Alternatively, one each of two second portions 76 may define the first and second ends 46, 48, respectively, and a first portion 74 may define the intermediate portion 50. This configuration may allow the ends 46, 48 of the pre-curved flexible member 14 to desirably conform to the anchor members 16. The construction of pre-curved flexible member 14 shown in FIG. 22 provides a cross-sectional flexibility gradient that is generally dependent upon the elastomeric characteristics of the first and second portions 74, 76.

FIG. 23 depicts another embodiment of pre-curved flexible member 14, which is similar to FIG. 22. Rather than two first portions 74 defining first and second ends 46, 48, respectively, two each of four first portions 74 substantially define the first and second ends 46, 48, respectively. To that end, each of the two first portions 74 of the first and second ends 46, 48 of FIG. 23 define opposing substantially semicircular portions, which together are situated only substantially circumferentially about the curved lengthwise central axis 54.

FIGS. 24 and 24A depict another embodiment of pre-curved flexible member 14, which is also similar to FIG. 22. Specifically, as best shown in FIG. 24A, the first portion 74 defines a half-pipe shape that extends along the length of curved central axis 54. The second portion 76 overlays the half-pipe shape and generally defines a hemispherical shape, whereby the first portion 74 substantially defines the first and second ends 46, 48 and the second portion 76 substantially defines the curved intermediate portion 50. Thus, the first and second ends 46, 48 substantially include a first material having a first elasticity and the curved intermediate portion 50 substantially includes a second material having a second elasticity greater than the first to provide the pre-curved flexible member 14 with variable flexibility. In an alternate embodiment, the second portion 76 defines the half-pipe shape and the first portion 74 overlays the half-pipe shape and generally defines the hemispherical shape. The pre-curved flexible member 14 further includes optional aperture 36 extending lengthwise through the pre-curved body 40 for receiving the connecting member 34.

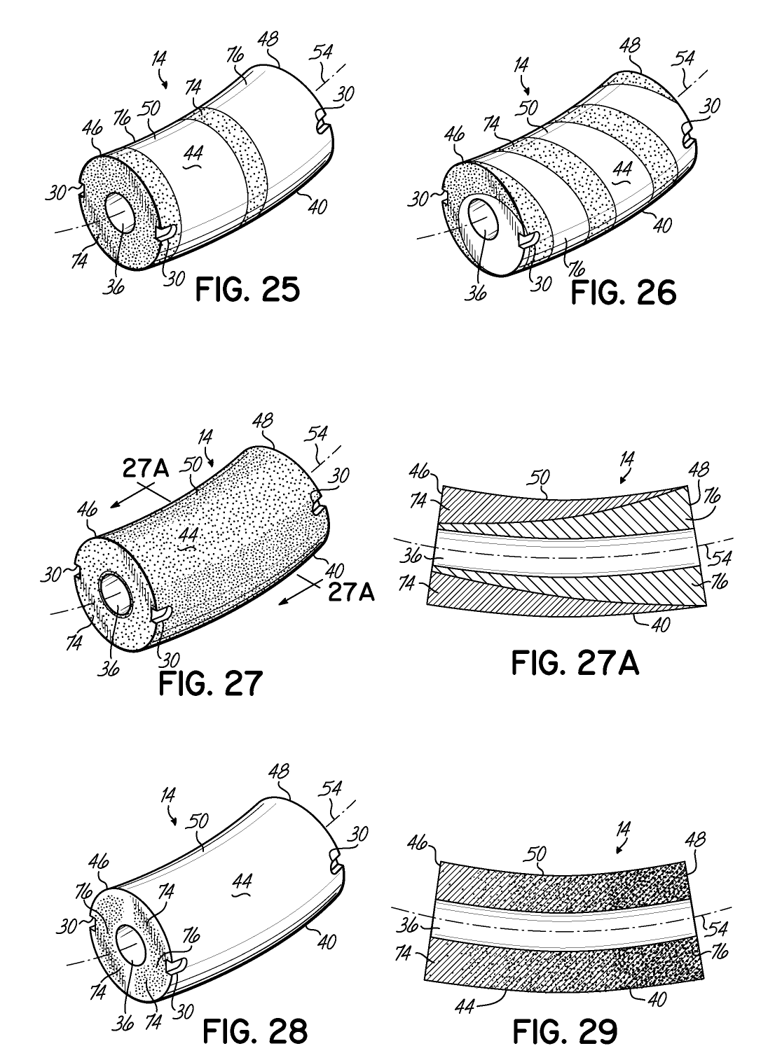

FIG. 25 depicts another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and plurality of first portions 74, i.e., two first portions, including a first material having a first elasticity and plurality of second portions 76, i.e., two second portions, including a second material having a second elasticity greater than the first to provide the pre-curved flexible member 14 with variable flexibility. The first and second portions 74, 76 are alternately situated circumferentially about the curved lengthwise central axis 54 along the length of the pre-curved body 40. The pre-curved flexible member 14 further includes optional aperture 36 extending lengthwise through the pre-curved body 40 for receiving connecting member 34. As shown, first end 46 is less flexible than second end 48, and the curved intermediate portion 50 has alternating regions of flexibility. In an alternate embodiment, the first and second portions 74, 76 may be switched such as to provide a variation in flexibility.

FIG. 26 depicts another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and first portion 74 including a first material having a first elasticity and second portion 76 including a second material having a second elasticity greater than the first to provide the pre-curved flexible member 14 with variable flexibility. The first portion 74 spirals along the length of the pre-curved body 40 with a remainder of the pre-curved body 40 including the second portion 76. As the spiral extends towards second end 48, the spacing between the turns of the spiral increases to provide the pre-curved flexible member 14 generally with a cross-sectional and lengthwise flexibility gradient. Other spiral variations may be provided. The pre-curved flexible member 14 further includes optional aperture 36 extending lengthwise through the pre-curved body 40 for receiving the connecting member 34.

As indicated above, with each pre-curved flexible member 14, the pre-curved flexible member 14 of FIG. 26, for example, may be provided in varying lengths, such as twelve-inch lengths, so that a surgeon can cut, or shape, the pre-curved flexible member 14 to fit between opposing anchor members 16 along a specific section of spine 10 as well as to accommodate a desired bending movement of the pre-curved flexible member 14. To that end, during surgery, a surgeon may cut pre-curved flexible member 14 of FIG. 26 to provide pre-curved flexible member 14 with a different bending property due to the spacing of the spiral, such spacing providing variable flexibility. The spiral configuration of the pre-curved flexible members 14 of FIG. 26 can allow for more consistent bending forces in all directions and can provide a desirable spring-action effect.

FIGS. 27 and 27A depict yet another embodiment of pre-curved flexible member 14, which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and first portion 74 including a first material having a first elasticity and second portion 76 including a second material having a second elasticity greater than the first to provide the pre-curved flexible member 14 with variable flexibility. As best shown in FIG. 27A, each of the first and second portions 74, 76 are generally triangular-shaped with each base situated at opposing ends 46, 48, respectively, such that first portion 74 substantially defines the first end 46 and second portion 76 substantially defines the second end 48. In other words, the first end 46 substantially includes the first material and the second end 48 substantially includes the second material. The pre-curved flexible member 14 further includes optional aperture 36 extending lengthwise through the pre-curved body 40 for receiving the connecting member 34. This configuration provides one end 46 that is less flexible than the other end 48. The pre-curved flexible member 14 in FIGS. 27 and 27A has a cross-sectional and lengthwise flexibility gradient.

FIG. 28 further depicts still another embodiment of pre-curved flexible member 14, which is similar to FIG. 20. However, the first and second materials of FIG. 28 includes the same type of polymer, e.g., polyurethane, wherein the first and second materials have a predetermined stoichiometry and thermal history, thereby providing the first and second elasticities, respectively, for the first and second portions. Such pre-curved flexible member with its material may be formed using the precision polyurethane manufacture method and apparatus disclosed in U.S. Patent Application Publication No. 2004/0049002 to Andrews et al., titled “Precision Polyurethane Manufacture”, which is expressly incorporated by reference herein in its entirety.

In the precision polyurethane manufacture process, a polyurethane having a predetermined stoichiometry and thermal history is generally formed by reacting a multifunctional isocyanate, a polyol and, optionally, a chain extender. At least two reagents selected from the isocyanate, the polyol, the chain extender, any mixture thereof and any pre-polymer formed therefrom are intensively mixed prior to being reactively extruded to form the polyurethane. The process is understood to allow control of reagent stoichiometry, while intimate mixing of the reagents at the molecular level allows the manufacture of tailored linear polyurethanes of narrow molecular weight distribution or narrow polydispersity. Moreover, a known and reproducible thermal history can be imparted to the polymer during synthesis, while overall thermal degradation can be minimized by reducing the number of melt cycles for polyurethane components. Such a process allows the integrated manufacture of a polyurethane resin, finished product or aqueous dispersion, while perturbations of the reaction stoichiometry allows the manufacture of polyurethanes with controlled composition and mass distributions.

With reference again to FIG. 28, the pre-curved flexible member 14 which includes pre-curved body 40 of a cylindrical shape having outer surface 44 and opposing ends 46, 48 with curved intermediate portion 50 extending therebetween. Each of the opposing ends 46, 48 includes a pair of notches 30 situated in opposing relation within the outer surface 44 adjacent a terminal edge of respective opposing ends 46, 48 for cooperation with corresponding anchor member retention features 20. The pre-curved body 40 also includes curved lengthwise central axis 54 and a plurality of first portions 74, i.e., two first portions, including a first material having a first elasticity and a plurality of second portions 76, i.e., two second portions, including a second material having a second elasticity greater than the first to provide the pre-curved flexible member 14 with a variable flexibility. The first and second portions 74, 76 extend along the length of the pre-curved body 40 and are alternately spaced symmetrically about the curved axis 54 of the pre-curved body 40. Again, the first and second materials include the same type of polymer, e.g., polyurethane formed by the precision polyurethane manufacture method discussed above, wherein the material has a predetermined stoichiometry and thermal history thereby providing the first and second elasticities, respectively, for the first and second portions 74, 76. The pre-curved flexible member 14 further includes optional aperture 36 extending lengthwise through the pre-curved body 40 for receiving the connecting member 34.

FIG. 29 depicts another embodiment of the pre-curved flexible member 14, which is similar to FIG. 28 insofar as the first and second materials of the pre-curved flexible member 14 include the same type of polymer, e.g., polyurethane, wherein the first and second materials having a predetermined stoichiometry and thermal history, thereby providing the first and second elasticities, respectively, for the first and second portions 74, 76. Such pre-curved flexible member 14 with its material may be formed using the precision polyurethane manufacture method and apparatus as discussed above.

To that end, the pre-curved flexible member 14 of FIG. 29 includes pre-curved body 40 of a cylindrical shape having outer surface 44 and opposing ends with curved intermediate portion extending therebetween. Each of the opposing ends 46, 48 includes a pair of notches 30 (not shown) situated in opposing relation within the outer surface 44 adjacent a terminal edge of respective opposing ends 46, 48 for cooperation with corresponding anchor member retention features 20. The pre-curved body 40 also includes curved lengthwise central axis 54 and first portion 74 including a first material having a first elasticity and second portion 76 including a second material having a second elasticity greater than the first to provide the pre-curved flexible member 14 with a variable flexibility. More specifically, the pre-curved body 40 of pre-curved flexible member 14 of FIG. 29 defines a flexibility gradient that extends along the length thereof. The flexibility gradient increases in flexibility from the first end 46 to the opposing second end 48 to define a plurality of elasticities, which includes the first and second elasticity for the first and second portions 74, 76, respectively. The first and second ends 46, 48 substantially define first and second portions 74, 76, which include first and second material respectively. That material has a predetermined stoichiometry and thermal history thereby providing the first and second elasticities, respectively, for the first and second portions 74, 76. In an alternate embodiment, the pre-curved body 40 can define a flexibility gradient that extends radially outward from the curved lengthwise central axis 54. Such flexibility gradient can increase or decrease in flexibility in a direction radially outwardly from and circumferentially about the lengthwise central axis 54. Other variations are contemplated and understood by one having ordinary skill in the art. The pre-curved flexible member 14 further includes optional aperture 36 extending lengthwise through the pre-curved body 40 for receiving the connecting member 34.

The materials that are used in the pre-curved flexible members 14 of the present invention may be selected from any suitable biocompatible material as known in the art. By way of example, the materials can include rigid or flexible metals, ceramic materials, carbon fiber, polymeric materials, and/or composite materials. The metals can include titanium or nickel-titanium alloy (NiTiNOL) wire, such as superelastic or shape memory NiTiNOL, for example. The polymeric materials can include, for example, hydrogels (e.g., polyacrylamides), silicone elastomers (natural or synthetic), epoxies (e.g., polyamide epoxy), urethanes, and thermoplastic materials, such as polyurethane, polyethylene (e.g., UHMWPE), polyethylene terephthalate (e.g., Sulene®), polypropylene, polyamide (e.g., Nylon), polyester, acetal, polycarbonate, thermoplastic elastomers, and the like. The composite materials may include, for example, resin impregnated graphite or aramid fibers (e.g., liquid crystal polymers such as Kevlar®), or NiTi dispersed in polyethylene terephthalate. The composite materials may be tailored to define a flexibility gradient, for example, by varying the type and/or amount of filler material therein, which may be controlled or metered during manufacture thereof. Such composite material, thus, can provide the pre-curved flexible member 14 with a flexibility gradient.

The selected second material generally includes an elasticity different than, either greater or lower than, the first material to provide the pre-curved flexible member 14 with a variable flexibility. Also, additional materials (third, fourth, fifth materials) with elasticities different than the first and second materials or the same as one of the first and second materials can be used. Determining the elasticity (or moduli of elasticity) of materials is well known in the art. It will be recognized that various other materials suitable for implantation of the pre-curved flexible member 14 within the human body and for providing stabilization of the spine while maintaining flexibility may be used.

The above-described pre-curved flexible members 14 can be manufactured using injection molding processes, or other suitable processes, as are known in the art. To that end, the proposed configurations may be injection molded using, for example, a one-step process or a multi-step process involving the material(s) of the pre-curved flexible member 14. In addition, the desired pre-curved flexible member 14 also may be extruded using a conventional thermoplastic extrusion process then heat formed to provide the desired curvature of the pre-curved body. Such process can utilize one or more extrusion heads having a die nozzle configuration to feed the materials into an extrusion die to form a well-fused combination of materials, i.e., to form the pre-curved flexible member 14.

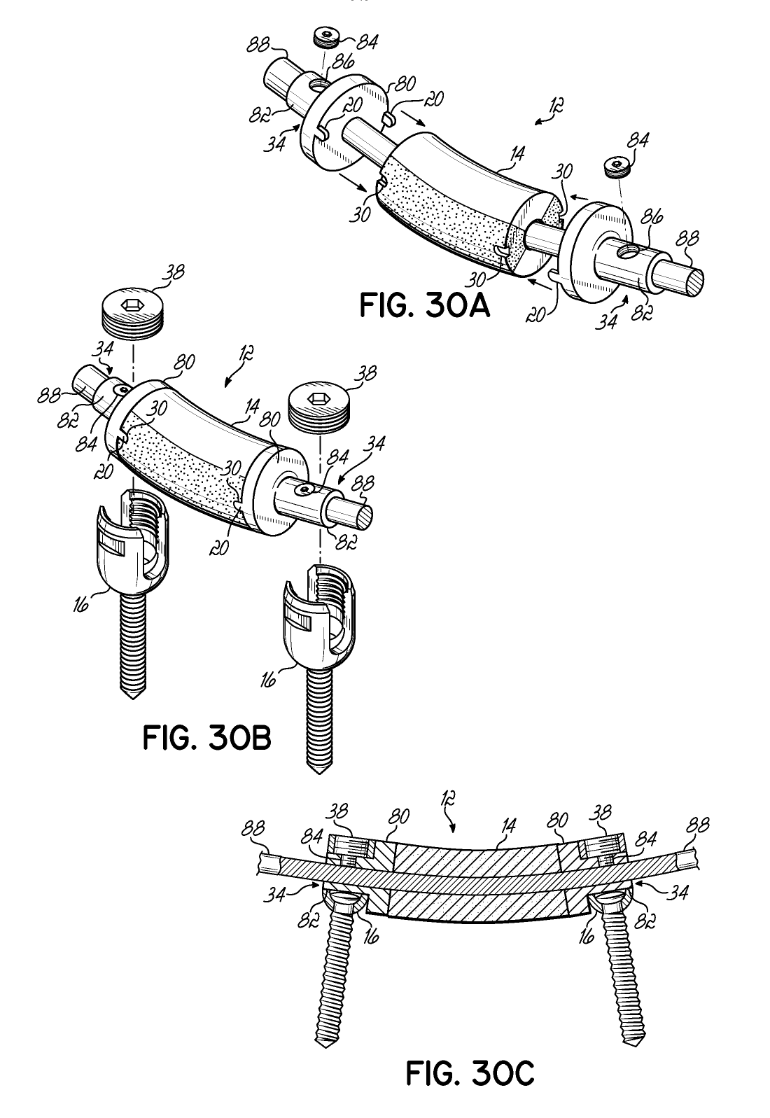

Referring now to FIGS. 30A-30C, an alternative embodiment of dynamic stabilization system or implant 12 is shown including pre-curved flexible member 14 of FIG. 20 positioned between anchor members 16. In this embodiment, the connecting members 34 include flanges 80 provided with outwardly projecting annular hubs 82 and a securing element in the form of a setscrew 84. The setscrew 84 is seated within a threaded aperture 86 on the hub 82 to secure the flange 80 and hub 82 arrangement to shank 88 of the system 12 and against the pre-curved flexible member 14. Each flange 80 includes protrusions 20, which generally extend in a direction away therefrom, for cooperating with corresponding notches 30 on the pre-curved flexible member 14 to securely retain pre-curved flexible member 14 between the anchor members 16 once the system is assembled. The system 12 can be assembled pre- or intra-operatively. Once assembled, the system 12 is positioned in the top loading anchor members 16 and secured thereto by the threadable cap members 38, as shown in FIG. 30B, resulting in the arrangement and installation of the pre-curved flexible member 14. A cross-sectional view of the system 12 and associated anchor members 16 of FIGS. 30A and 30B is shown in FIG. 30C.

Accordingly, there is provided pre-curved flexible member 14 with retention features, and which may also have variable flexibility, for use with a dynamic stabilization system 12 to provide dynamic stability to a person's spine 10. The retention features 20, 24, 30, 32 help retain the pre-curved flexible member 14 between the anchor members 16 at a desired orientation and help the pre-curved flexible member 14 maintain that desired orientation. In addition, such variability in flexibility, for example, can provide surgeons with further greater options in selecting the most appropriate pre-curved flexible member 14 for placement at a specific location along the spine 10, such selection being dictated by the desired bending movement of the pre-curved flexible member 14 at that location.

While the invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. For example, one or more characteristics of the above described pre-curved flexible members may be combined to give yet additional embodiments. Thus, the invention in its broader aspects is therefore not limited to the specific details, representative apparatus and/or method, and illustrative examples shown and described. Accordingly, departures may be made from such details without departing from the scope of applicant's general inventive concept.

What is claimed is:

1. A flexible member for use in stabilizing a spinal column comprising:
a pre-curved body including an outer surface and opposing ends with a curved intermediate portion extending therebetween, each of the opposing ends comprising a retention feature for cooperation with an anchor member to limit rotation of the pre-curved body, wherein the retention feature at each of the respective opposing ends is selected from one or a combination of:
(a) at least one notch provided within the outer surface of the respective opposing end; and/or
(b) at least one protrusion extending in a direction away from the respective opposing end.

2. The flexible member of claim 1 wherein the retention feature at each of the respective opposing ends is selected from (a), the at least one notch including a pair of notches situated in opposing relation and within the outer surface of the respective opposing end.

3. The flexible member of claim 1 wherein the retention feature at each of the respective opposing ends is selected from (b), the at least one protrusion including a pair of protrusions situated in opposing relation and extending in the direction away from the respective opposing end.

4. The flexible member of claim 1 wherein the at least one protrusion extends parallel with a curved lengthwise axis of the body.

5. The flexible member of claim 1 wherein the at least one notch is provided within the outer surface adjacent a terminal edge of the respective opposing end.

6. The flexible member of claim 1 wherein the pre-curved body includes more than one curve along its length.

7. The flexible member of claim 1 wherein the curved intermediate portion comprises a first groove within the outer surface to provide the flexible member with a variable flexibility.

8. The flexible member of claim 1 wherein the curved intermediate portion comprises a plurality of grooves within the outer surface to provide the flexible member with a variable flexibility.

9. The flexible member of claim 1 wherein the body comprises a polymeric or metallic material.

10. The flexible member of claim 1 wherein the body includes an aperture extending lengthwise therethrough.

11. The flexible member of claim 1 further including a taper in diameter of the pre-curved body.

12. The flexible member of claim 1 wherein the pre-curved body further includes at least one first portion comprising a first material having a first elasticity and at least one second portion comprising a second material having a second elasticity greater than the first to provide the flexible member with a variable flexibility.

13. The flexible member of claim 12 wherein the first material comprises a different polymer than the second material.

14. The flexible member of claim 12 wherein the first and second materials comprise a polymer of the same type, with the first and second materials having a different predetermined stoichiometry and thermal history thereby providing the first and second elasticities, respectively, for the first and second portions.

15. The flexible member of claim 14 wherein the pre-curved body defines a flexibility gradient that extends along the length thereof, the flexibility gradient increasing in flexibility from the first end to the opposing second end to define a plurality of elasticities, which includes the first and second elasticity for the at least one first and second portions.

16. A method for stabilizing a patient's spine with an implant, comprising the steps of:
providing a plurality of implants, each of the implants comprising a flexible member having a pre-curved body including an outer surface and opposing ends with a curved intermediate portion extending therebetween, each of the opposing ends comprising a retention feature for cooperation with an anchor member to limit rotation of the pre-curved body, wherein the retention feature at each of the respective opposing ends is selected from one or a combination of:
(a) at least one notch provided within the outer surface of the respective opposing end; and/or
(b) at least one protrusion extending in a direction away from the respective opposing end;
selecting at least one implant from the plurality of implants based on the condition of the patient; and
implanting the selected implant into the patient.

17. The method of claim 16, wherein each flexible member has a variable flexibility measured over a cross-section of the flexible member.
